(19) United States
(12) Patent Application Publication
(10) Pub. No.: US 2019/0328374 A1
(43) Pub. Date: Oct. 31, 2019

(54) TRANS-SEPTAL CLOSURE DEVICE

(71) Applicant: Edwards Lifesciences Corporation, Irvine, CA (US)

(72) Inventor: Carey Philip Hendsbee, San Clemente, CA (US)

(21) Appl. No.: 16/286,108

(22) Filed: Feb. 26, 2019

Related U.S. Application Data
(60) Provisional application No. 62/662,351, filed on Apr. 25, 2018.

(51) Int. Cl. A61B 17/00

(52) U.S. Cl. CPC A61B 17/0057; A61B 2017/00575; A61B 2017/00867; A61B 2017/00243; A61B 2017/00004

(57) ABSTRACT

An implantable closure device can comprise a self-expanding metal frame comprising a central portion defining a lumen and a plurality of first anchoring arms and a plurality of second anchoring arms angularly spaced around the central portion. The central portion can comprise a sinusoidal-shaped ring defining a plurality of apices. The first anchoring arms can be connected to locations inside of the apices on a first side of the central portion and the second anchoring arms can be connected to locations inside of the apices on a second side of the central portion. The first anchoring arms can be configured to bear against a first surface of a septal wall. The second anchoring arms can be configured to bear against a second surface of the septal wall.

### CROSS REFERENCE TO RELATED APPLICATION

This application claims the benefit of U.S. Provisional Application Ser. No. 62/662,351, filed Apr. 25, 2018, which is incorporated herein by reference.

### FIELD

The present disclosure relates generally to a method and device for closing a septal defect, or opening in a septum. In particular, the present disclosure relates to a method and device for closing a septal defect, for example a defect in an atrial septum, such that the septal defect can be accessed for reentry through the defect.

### BACKGROUND

A septum may include a thin wall dividing a cavity into two smaller structures. An atrial septum is a wall of tissue separating the left and right atria of the heart. A ventricular septum is a wall of tissue separating the left and right ventricles of the heart. A septal defect may include a perforation or hole passing through the septum. A septal defect can occur congenitally or by puncturing the septum with a medical device to access a location within the heart.

The atrial septum may be viewed like the femoral artery in years to come. The femoral artery is an access point for many catheterization laboratory procedures, with a smaller percentage of procedures utilizing venous or radial artery access. Likewise, the atrial septum is a point of percutaneous access for atrial fibrillation therapy, left atrial appendage closure, percutaneous mitral valve reset, and percutaneous mitral valve replacement. In these and other procedures, devices may traverse across the atrial septum and, by doing so, may leave a defect or orifice in the atrial septum that cannot close spontaneously. Currently, these defects are closed using devices, such as plugs, that may close the defect but do not allow for re-access through the septum. Thus a need exists for improved closure devices for closing a septal defect and for re-accessing the left side of the heart in subsequent procedures.

### SUMMARY

Embodiments of an implantable closure device are disclosed herein, as well as related methods and apparatuses including such closure devices. In several embodiments, the disclosed closure devices are configured to close a septal defect.

In one representative embodiment, an implantable closure device can comprise a self-expanding metal frame comprising a central portion defining a lumen and a plurality of first anchoring arms and a plurality of second anchoring arms angularly spaced around the central portion. The central portion can comprise a sinusoidal-shaped ring defining a plurality of apices. The first anchoring arms can be connected to locations inside of the apices on a first side of the central portion and the second anchoring arms can be connected to locations inside of the apices on a second side of the central portion. The first anchoring arms can be configured to bear against a first surface of a septal wall. The second anchoring arms can be configured to bear against a second surface of the septal wall.

In some embodiments, the closure device can have at least two first anchoring arms and at least two second anchoring arms. In some embodiments, the sinusoidal-shaped ring can comprise a plurality of angled struts interconnected by the plurality of apices. In some embodiments, the sinusoidal-shaped ring can comprise a first set of apices connected to the first anchoring arms, a second set of apices connected to the second anchoring arms, and a third set of apices that are not connected to either the first anchoring arms or the second anchoring arms. In some embodiments, at least one apex of the third set of apices can be positioned circumferentially between each first anchoring arm and a second anchoring arm.

In some embodiments, the sinusoidal-shaped ring can comprise a number of apices equal to the total number of first anchoring arms and second anchoring arms. In some embodiments, the closure device can include an occluding member mounted on the frame and at least partially covering the lumen of the central portion. In some embodiments, the occluding member can be configured to be pierced by a medical instrument. In some embodiments, the occluding member can comprise a fabric.

In another representative embodiment, an implantable closure device can comprise a self-expanding metal frame comprising a central portion defining a lumen and a plurality of first anchoring arms and a plurality of second anchoring arms angularly spaced around the central portion. The first anchoring arms and the second anchoring arms can each have tissue engaging surfaces. The first anchoring arms and the second anchoring arms can be configured to move from a non-deflected state prior to implantation in a septum to a deflected state when implanted in the septum. When the anchoring arms are in the non-deflected state, the tissue engaging surfaces of the first anchoring arms and the tissue engaging surfaces of the second anchoring arms can face away from each other and when the anchoring arms are in the deflected state, the tissue engaging surfaces of the first anchoring arms and the tissue engaging surfaces of the second anchoring arms can contact opposing sides of the septum.

In some embodiments, the closure device can have at least two first anchoring arms and at least two second anchoring arms. In some embodiments, the central portion can comprise a sinusoidal-shaped ring defining a plurality of apices and each anchoring arm can extend from one of the apices.

In some embodiments, each anchoring arm can have a first portion extending from the central portion generally parallel to a central axis of the closure device and a second portion extending radially outwardly from the first portion. In some embodiments, the closure device can comprise a first set of apices connected to the first anchoring arms, a second set of apices connected to the second anchoring arms, and a third set of apices that are not connected to either the first anchoring arms or the second anchoring arms. In some embodiments, the closure device can have an occluding member mounted to the frame and at least partially covering the lumen of the central portion.

In another representative embodiment, a method of implanting a closure device can comprise coupling the closure device to a distal end portion of a delivery apparatus, inserting the distal end portion of the delivery apparatus and the closure device into a patient's body, and positioning the central portion of the closure device within a septal orifice of a septal wall. The closure device can comprise a self-expanding metal frame comprising a central portion defining a lumen and a plurality of first anchoring arms and a plurality of second anchoring arms angularly spaced around the central portion. The first anchoring arms and the second anchoring arms can each have tissue engaging surfaces. The frame can have a non-deflected state in which the tissue engaging surfaces of the first anchoring arms and the tissue engaging surfaces of the second anchoring arms can face away from each other. The method can further include positioning the first anchoring arms and the second anchoring arms such that the tissue engaging surfaces of the first anchoring arms engage a first side of the septal wall and the tissue engaging surfaces of the second anchoring arms engage a second side of the septal wall such that the first and second anchoring arms are held in a deflected state.

In some embodiments, the closure device can be held in a radially compressed state when inserted into the patient's body and can be radially expanded prior to or during the acts of positioning. In some embodiments, the central portion can comprise a sinusoidal-shaped ring defining a plurality of apices and each anchoring arm can extend from the inside of one of the apices.

In some embodiments, the first anchoring arms can be spaced from the second anchoring arms by a gap when the anchoring arms are in the non-deflected state and the gap can be reduced when the anchoring arms are in the deflected state. In some embodiments, the first and second anchoring arms can be substantially co-planar with each other in the deflected state.

The foregoing and other objects, features, and advantages of the present disclosure will become more apparent from the following detailed description, which proceeds with reference to the accompanying figures.

### BRIEF DESCRIPTION OF THE DRAWINGS

FIGS. 1A and 1B are top views of a septal closure device, according to one embodiment.

FIG. 2 is a flattened view of a portion of the septal closure device of FIG. 1.

FIG. 3 is a perspective view of the septal closure device of FIG. 1.

FIG. 4 is a side view of the septal closure device of FIG. 1.

FIG. 5 is a top view of a septal closure device, according to another embodiment.

FIG. 6 is a flattened view of the septal closure device of FIG. 5.

FIG. 7 is a perspective view of the septal closure device of FIG. 5.

FIG. 8 is a side view of the septal closure device of FIG. 5.

FIG. 9 is a top view of a septal closure device, according to another embodiment.

FIG. 10 is a flattened view of the septal closure device of FIG. 9.

FIG. 11 is a perspective view of the septal closure device of FIG. 9.

FIG. 12 is a top view of a septal closure device, according to another embodiment.

FIG. 13 is a flattened view of the septal closure device of FIG. 12.

FIG. 14 is a perspective view of the septal closure device of FIG. 12.

FIG. 15 is a top view of a septal closure device, according to another embodiment.

FIG. 16 is a flattened view of the septal closure device of FIG. 15.

FIG. 17 is a perspective view of the septal closure device of FIG. 15.

FIG. 18 is a side view of the septal closure device of FIG. 15.

FIG. 19 is a top view of a septal closure device, according to another embodiment.

FIG. 20 is a flattened view of the septal closure device of FIG. 19.

FIG. 21 is a perspective view of the septal closure device of FIG. 19.

FIG. 22 is a side view of the septal closure device of FIG. 19.

FIG. 23 is a top view of a septal closure device, according to another embodiment.

FIG. 24 is a flattened view of the septal closure device of FIG. 23.

FIG. 25 is a perspective view of the septal closure device of FIG. 23.

FIG. 26 is a side view of the septal closure device of FIG. 23.

FIGS. 27A and 27B area perspective views of a septal closure device implanted in a septum of a patient.

FIGS. 28A and 28B are a top view and a side view, respectively, of a septal closure device according to another embodiment.

FIGS. 29-31 show various stages of an implantation procedure of the septal closure device of FIGS. 28A and 28B.

FIG. 32 shows a top view of the septal closure device of FIGS. 28A and 28B in an implanted position.

FIG. 33 shows a side view of the septal closure device of FIGS. 28A and 28B in an implanted position.

### DETAILED DESCRIPTION

In certain embodiments, a septal closure device is suitable to close or reset a septal orifice and allow for re-entry through a septum at the same septal orifice location at a later time as other therapeutic interventions are warranted. In certain embodiments, the closure device is suitable to provide an access port for accessing the left side of the heart with a catheter or other medical device. As used herein, the term “septal orifice” or “orifice” is used to describe an orifice created by puncturing the septum with a catheter or other medical device and an orifice that occurs congenitally, such as an atrial septal defect (ASD) or a patent foramen ovale (PFO).

The embodiments of the closure device described below are described in the context of occluding or closing an orifice in the atrial septum. The disclosed embodiments also can be implanted in orifices formed in a ventricular septum, the apex or other sections of the heart, or in orifices (surgically or congenitally formed orifices) formed in other organs of the body.

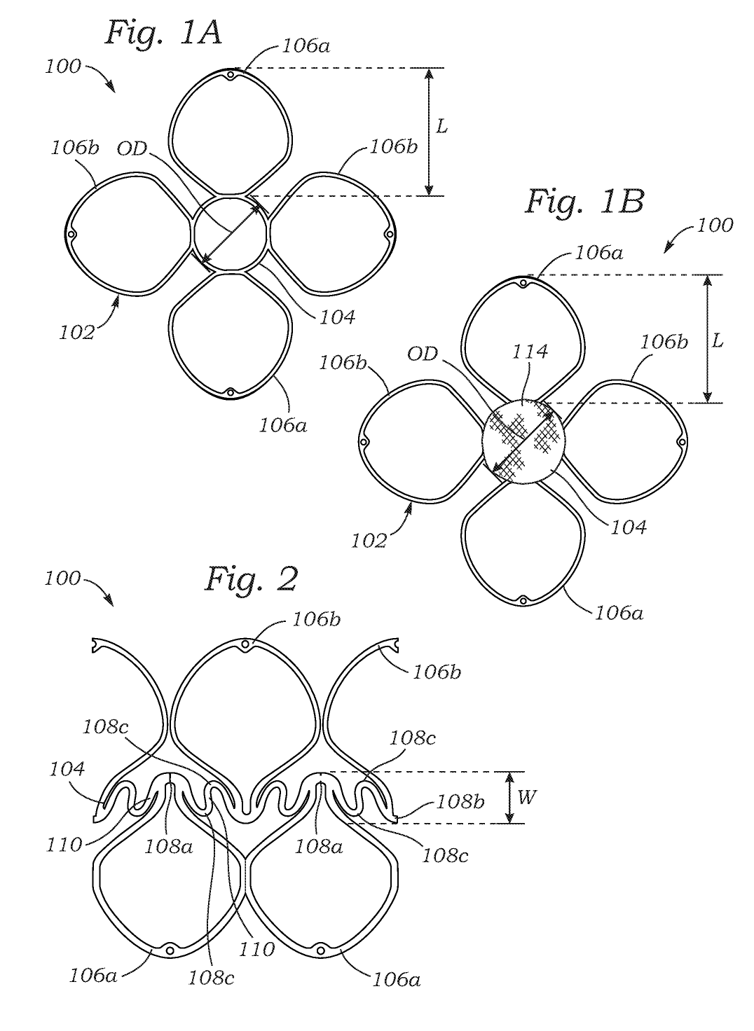

As shown in FIG. 1A, a septal closure device 100 can comprise a frame 102 comprising a central portion 104, a first set of anchoring arms 106a, and a second set of anchoring arms 106b. The first set of anchoring arms 106a and the second set of anchoring arms 106b can be angularly spaced around the central portion 104. In the illustrated example of FIG. 1A, the frame 102 comprises four anchoring arms including two first anchoring arms 106a and two second anchoring arms 106b. In other examples, the frame can comprise a fewer or greater number of anchoring arms. In the illustrated example of FIG. 1A, the anchoring arms 106a, 106b are loop-shaped or petal-shaped structures. In other examples, the anchoring arms can have any other shape. In some examples, the anchoring arms can comprise elongated wires or strut members that are secured to the central portion 104 at only one end of the wire or strut member. In particular embodiments, the central portion 104 can have an outer diameter OD in the range of about 5 mm to about 15 mm, and more particularly in the range of about 7 to about 10 mm, with 8 mm being a specific example. In particular embodiments, the anchoring arms 106a, 106b can each have a length L in the range of about 5 mm to about 15 mm, and more particularly in the range of about 9 mm to about 13 mm, and even more particularly in the range of about 11 mm to about 12 mm.

When implanted in a patient, the central portion 104 can be positioned within the septal orifice that is to be closed. The anchoring arms can extend radially outwardly from the central portion 104 and perpendicularly or substantially perpendicularly to a central axis of the closure device 100 such that an atrial septum can be compressed or pinched between the anchoring arms as discussed in further detail below. As shown in FIG. 1B, an occluding member 114 can be mounted over the central portion 104 prevent blood from flowing through the device, thus closing the orifice. The occluding member 114 can comprise any of various bioresorbable or non-bioresorbable materials, as discussed further below.

In some examples, the occluding member can be pierced by a catheter or another medical instrument to re-access the left side of the heart in a future procedure. If the medical instrument has a relatively small diameter, such as used for treating arrhythmias, the hole formed in the occluding member and/or regrown tissue may be small enough to sufficiently inhibit blood flow between the left and right atriums without further intervention. If the medical instrument has a relatively large diameter, such as a delivery apparatus for implanting a prosthetic valve, and leaves a relatively larger opening in the occluding member and/or regrown tissue, another closure device can be implanted within the first closure device 100 to block blood flow between the right and left atriums.

Various types of medical instruments can be passed through the closure device 100 to access the left side of the heart. The medical instrument can be, for example, a delivery apparatus for delivering and implanting a prosthetic heart valve in the native mitral valve or the native aortic valve. In alternative embodiments, the delivery apparatus can be used to deliver and implant various other prosthetic devices in the left atrium, mitral valve, left ventricle, and/or the aortic valve, including, for example, annuloplasty rings, closure devices for the left atrial appendage, sealing devices or reshaping devices for resetting or reshaping portions of the heart. In other embodiments, other percutaneous medical instruments can be advanced through the closure device 100 for performing a procedure on the left side of the heart, such as atrial fibrillation therapy.

In some examples, the occluding member 114 can comprise one or more pieces of bioresorbable material, film or cloth that are configured to encourage tissue ingrowth and can degrade over time, leaving just regrown tissue within the central portion 104. For example, the occluding member can comprise one or more pieces of bioresorbable electro-spun polymeric material, such as polylactide (PLA), polylactide glycolides (PLGA), polycaprolactone (PLC), polyacrylonitrile (PAN), poly(lactide-co-caprolactone) (PLCL), polygyconate, and polypeptides. Compared to woven fabrics, electro-spun polymers promote faster tissue ingrowth, have faster biodegradation times, are potentially less thrombogenic, and can be created weaker and therefore can be easily punctured with a medical instrument during subsequent re-crossing of the closure device.

In other embodiments, the occluding member 114 can comprise one or more sheets of pieces of non-bioresorbable material, such as any of various synthetic fabrics (e.g., polyethylene terephthalate (PET)) or natural tissue (e.g., pericardium). In some embodiments, the occluding member can be completely or substantially impermeable to blood. In other embodiments, the occluding member can be semi-porous to blood flow (e.g., a porous fabric). The porous material can be selected to remain porous or to close up and become impermeable or non-porous to blood over time. In a specific implementation, the occluding member can be made of a bio-spun polyurethane having a fiber size between 0.05 to 1.5 microns and a porosity of between 50% and 80%. The thickness of the occluding member can be between 100 to 200 microns. In another implementation, the occluding member can be made of a bio-spun polymer blend comprising polyurethane and PET, such as a 70/30% blend of polyurethane/PET, having similar fiber sizes and porosity.

In still alternative embodiments, the occluding member can be made of a biocompatible foam, such as polyurethane, PET, silicone, or polyethylene foam.

The occluding member can, but need not create a fluid-tight seal with the adjacent tissue of the septum, and instead can, at least initially, permit a small amount of blood flow between the atria (referred to as residual shunting). Over time, the occluding member can promote tissue ingrowth and completely close the orifice and prevent residual shunting between the atria. The occluding member can completely cover the lumen of the central portion 104 or the occluding member can cover a portion of the lumen of the central portion 104. As noted above, the occluding member can be configured such that the septal defect can be accessed for reentry through the defect either before or after occluding member degradation.

The occluding member 114 can be attached to the frame 102 (e.g., the central portion 104) via heat staking, sutures, molding, bonding, weaving and/or other techniques or mechanisms known to those skill in the art with the benefit of the present disclosure. For example, the outer edges of the occluding member 114 can be folded over the central portion 104 and then welded to a more central area of the occluding member to fix the occluding member to the frame 102. The occluding member may extend beyond the periphery of the central portion 104, for example up to 2 mm. In some embodiments, the occluding member may have a generally circular shape prior to attachment to the frame 102.

The occluding member can include a plurality of notches that align with the anchoring arms 106a, 106b to aid in the folding of the occluding member over the periphery of the central portion 104. Additionally and/or alternatively, the occluding member may have any shape configured to cover all or a portion of the lumen of a central portion, as known to those skilled in the art with the benefit of the present disclosure.

In particular embodiments, the occluding member 114 can be configured to block the flow of blood between the right and left atriums through the closure device 100 and optionally can permit passage of a medical device through the lumen of the closure device 100. For an adult, the normal range of right atrial pressure (RAP) is about 2-6 mmHg and the normal range of left atrial pressure (LAP) is about 4-12 mmHg Thus, throughout most of the cardiac cycle, the LAP is greater than the RAP. In some embodiments, the occluding member 114 can be configured to block at least the flow of blood from left atrium to the right atrium. In other embodiments, the occluding member 114 can be configured to block the flow of blood between the right and left atriums in both directions throughout the cardiac cycle.

In certain embodiments, for example, the occluding member 114 can comprise a valve, such as in the form of a plurality of leaflets or flaps that are arranged relative to each other to maintain a closed position against a blood pressure gradient between the right atrium and the left atrium but can be opened by the force of a catheter or other medical instrument to permit passage of the medical instrument through the lumen of the closure device 100. The flaps primarily block the flow of blood from the left atrium to the right atrium due to the typically higher LAP, but can also block the flow of blood from the right atrium to the left atrium if the RAP exceeds the LAP. An occluding member in the form of a valve is further described in co-pending U.S. Publication No. 2017/0224323, which is incorporated herein by reference. The closure device 100 can incorporate any of the occluding members disclosed in U.S. Publication No. 2017/0224323.

The frame 102 can be self-expandable and can be formed from a shape-memory material, such as Nitinol, so that the frame self-expands from a delivery configuration to a deployed configuration when released or deployed from a delivery apparatus. In alternative embodiments, the frame 102 can be formed from a plastically-expandable material, such as stainless steel or cobalt-chromium alloy, and can be configured to be plastically expanded from a delivery configuration to a deployed configuration by an expansion device, such as an inflatable balloon.

In particular embodiments, the frame 102 is laser cut or otherwise formed from a tubular piece of metal, such as Nitinol, and the anchoring arms are bent away from the central portion and shape set in the configuration shown in FIGS. 1A, 1B, 3 and 4. Alternatively, the frame 102 can be laser cut or otherwise formed from a flat sheet of metal, such as Nitinol, and then shape set in the configuration shown in FIGS. 1A, 1B, 3 and 4. Alternatively, the frame 102 can be formed by bending one or more metal wires into the form shown.

FIG. 2 shows a flattened or unrolled view of the septal closure device 100 of FIG. 1A. As can be seen in FIG. 2, the central portion 104 comprises a sinusoidal shaped ring comprising a plurality of struts 110 interconnected by a first set of apices 108a, a second set of apices 108b, and a third set of apices 108c. Each of the first anchoring arms 106a can extend from a corresponding first apex 108a and each of the second anchoring arms 106b can extend from a corresponding second apex 108b. In the illustrated example, there are two apices 108c between each first apex 108a and the closest second apex 108b in the circumferential direction. In other examples, there can be any number of apices 108c between each first apex 108a and the closest second apex 108b. The first set of anchoring arms 106a can extend from one side of the central portion 104 (down in the orientation of FIG. 2) and the second set of anchoring arms 106b can extend from the other side of the central portion (up in the orientation FIG. 2). In other examples, the central portion 104 can be shaped in ways other than sinusoidally.

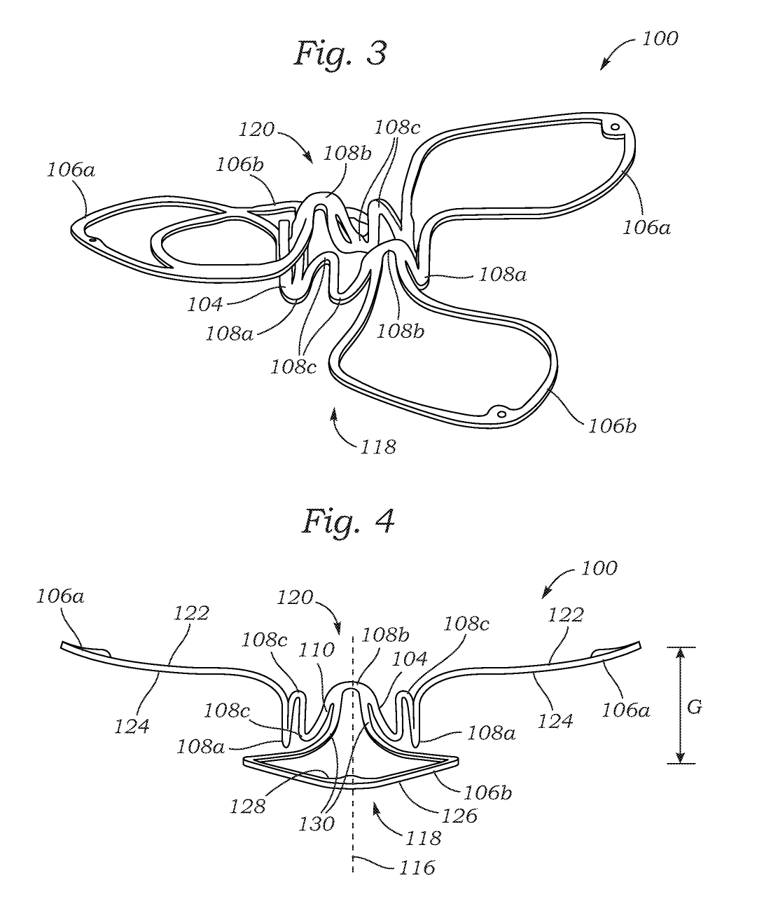

The central portion 104 can be radially compressed to a radially compressed configuration for delivery to an implantation site within the sheath of a delivery apparatus. If formed from a shape-memory material (e.g., Nitinol), the central portion 104 can self-expand to the radially expanded configuration shown in FIGS. 3-4. Moreover, due to its sinusoidal shape, the central portion 104 can further expand radially to accommodate a medical device having a larger diameter that is inserted through the central portion in a subsequent medical procedure after the closure device 100 is implanted.

In some examples, the central portion 104 can have an axial width W in the range of about 1 mm to about 2 mm, and more particularly in the range of about 1.2 mm to about 1.6 mm, with 1.4 mm being a specific example.

As can best be seen in FIGS. 3 and 4, apices 108a are positioned on a first side 118 of the central portion 104 (the bottom in the orientation of FIGS. 3 and 4) and apices 108b are positioned on a second side 120 of the central portion (the top in the orientation of FIGS. 3 and 4). Each anchoring arm can be connected to the underside or inside of the apex (i.e., the concave side of the apex). As such, in the orientation of FIG. 3, the first set of anchoring arms 106a extend from apices 108a on the first side 118 of the central portion 104 to a position on the second side 120 of the central portion. Likewise, the second set of anchoring arms 106b extend from apices 108b on the second side 120 of the central portion 104 to a position on the first side 118 of the central portion. In the illustrated embodiment, each anchoring arm initially extends generally parallel to a central axis 116 of the central portion 104 from a corresponding apex on one side of the central portion to the other side of the central portion, and then bends and extends radially outwardly from the central portion in a generally perpendicular relationship with respect to the central axis 116. Each of the anchoring arms 106a has a tissue engaging surface 122 and an opposing surface 124. Each of the anchoring arms 106b has a tissue engaging surface 126 and an opposing surface 128.

When delivered to an implantation site within a patient's body, the closure device 100 can be held in a radially compressed state within a sheath of a delivery apparatus. FIGS. 3 and 4 show the closure device 100 is an expanded configuration when deployed from the sheath of the delivery apparatus. When implanted in a septal orifice of a patient, each anchoring arm can be bent back toward its corresponding apex on the central portion 104 and held in a deflected state engaging the septal wall on the same side of the septal wall as where the apex is located. More specifically, the anchoring arms 106a are bent back toward apices 108a on the first side 118 of the central portion 104 such that tissue engaging surfaces 122 press against the septal wall. Likewise, the anchoring arms 106b are bent back toward the apices 108b such that tissue engaging surfaces 126 press against the opposite side of the septal wall.

In this manner, in the expanded, non-deflected state, the radially extending portions of the anchoring arms 106a are initially axially spaced from the radially extending portions of anchoring arms 106b to define a gap G (as shown in FIG. 4). When implanted within a septal wall, the anchoring arms are held in a deflected state in which the radially extending portions of the anchoring arms 106a are held closer to the radially extending portions of the anchoring arms 106b such that the gap G is reduced or alternatively, the anchoring arms 106a are substantially co-planar with the anchoring arms 106b such that the gap G eliminated. The opposing forces of the anchoring arms hold the closure device 100 securely in place within the septal wall.

In the illustrated example of FIGS. 1-4, anchoring arms 106a, 106b each have the same shape and are equally spaced angularly around the central portion 104 so as to not overlap with each other in the circumferential direction of the closure device 100; that is, a line extending parallel to the central axis 116 does not extend through or intersect more than one anchoring arm. In other examples, the anchoring arms can have different shapes and can have different angular spacing around the central portion with respect to each other. In some examples, the anchoring arms can overlap each other in the circumferential direction; that is, a line extending parallel to the central axis 116 can extend through or intersect two or more anchoring arms. The anchoring arms can have various shapes, including, but not limited to, a circle, a square, an ellipse, a diamond, a rectangle, an oval, or combinations thereof.

In some embodiments, the anchoring arms can be partially or fully covered with a cover that extends over the openings of the anchoring arms to facilitate tissue ingrowth and/or reduce trauma with the tissue contacting the anchoring arms. The cover can be separate pieces of material, with each one secured to a respective anchoring arm and sized and shaped to cover the opening in the anchoring arm. In other embodiments, the cover can be a single piece of material that is sized and shaped to cover all of the anchoring arms. In some embodiments, the cover can also include the occluding member 114. The cover can be made from any of the materials discussed above for the occluding member 114.

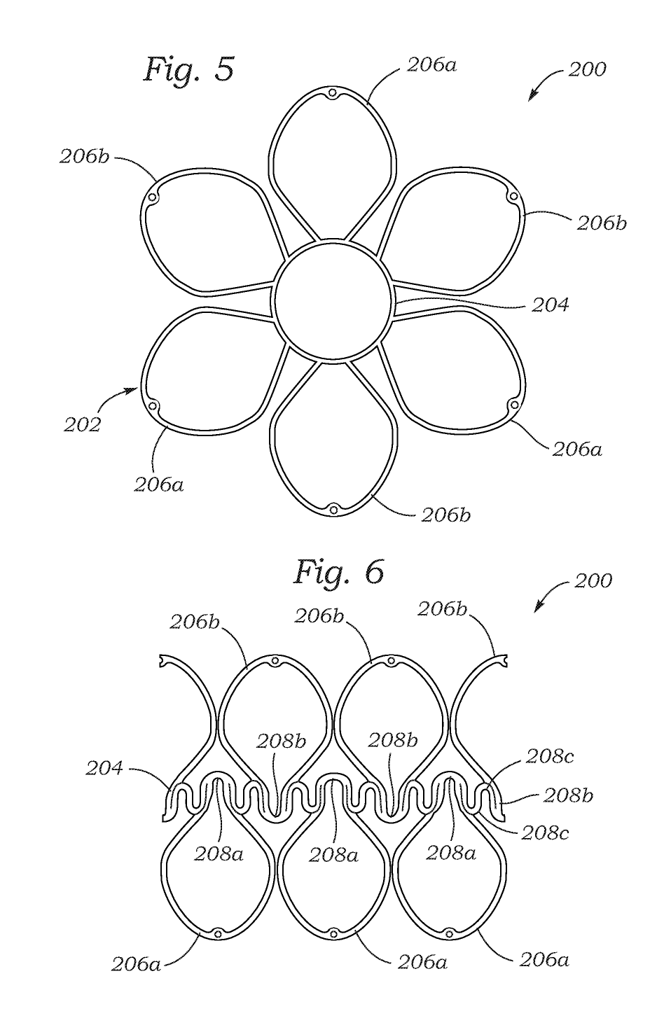

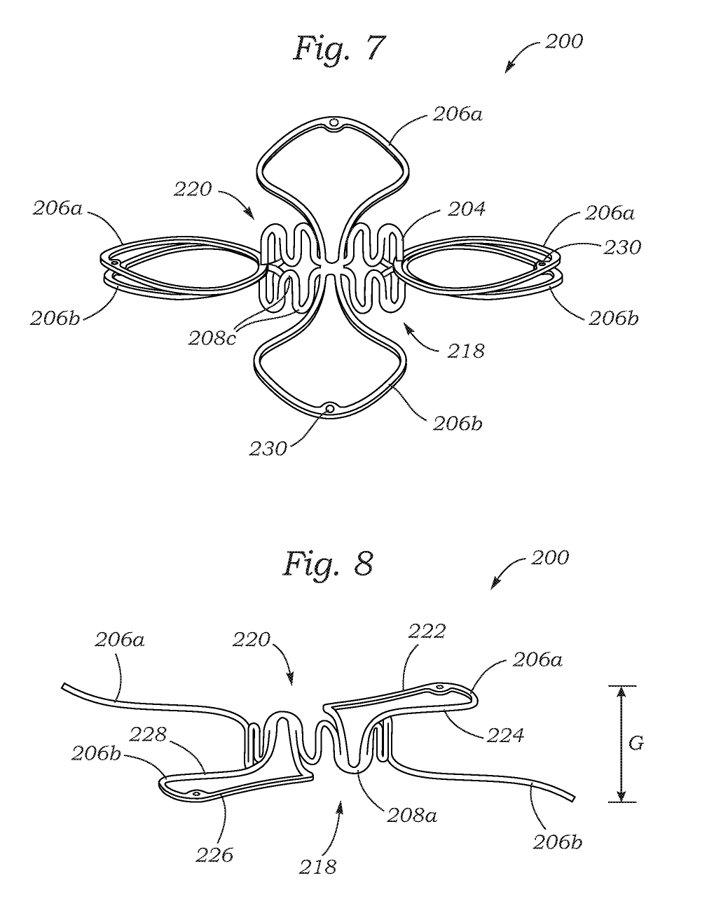

FIGS. 5-8 show an exemplary septal closure device 200, according to another embodiment. The septal closure device 200 comprises a frame 202 having a central portion 204, a first set of anchoring arms 206a, and a second set of anchoring arms 206b. Although not shown, an occluding member (e.g., occluding member 114) can be mounted on the frame 202 as described above in connection with closure device 100. The closure device 200 is constructed in a similar manner to the closure device 100 except that the closure device 200 has four anchoring arms in the first set of anchoring arms 206a and four anchoring arms in the second set of anchoring arms 206b. As can be seen in FIG. 6, the central portion 204 has a sinusoidal shape similar to the central portion 104. Each anchoring arm 206a, 206b extends from a respective apex 208a, 208b of the central portion 204. In the illustrated example, there are two apices 208c between each apex 208a and the closest apex 208b in the circumferential direction. In other examples, there can be any number of apices 208c between each apex 208a and the closest apex 208b. As can best be seen in FIGS. 7 and 8, the anchoring arms 206a extend from a first side 218 of the central portion 204 to a second side 220 of the central portion while anchoring arms 206b extend from the second side 220 of the central portion to the first side 218 of the central portion. Each anchoring arm 206a has a tissue engaging surface 222 and an opposing surface 224. Each anchoring arm 206b has a tissue engaging surface 226 and an opposing surface 228. In the expanded, non-deflected state shown in FIGS. 7 and 8, the tissue engaging surfaces 222 face in an opposite direction from the tissue engaging surfaces 226.

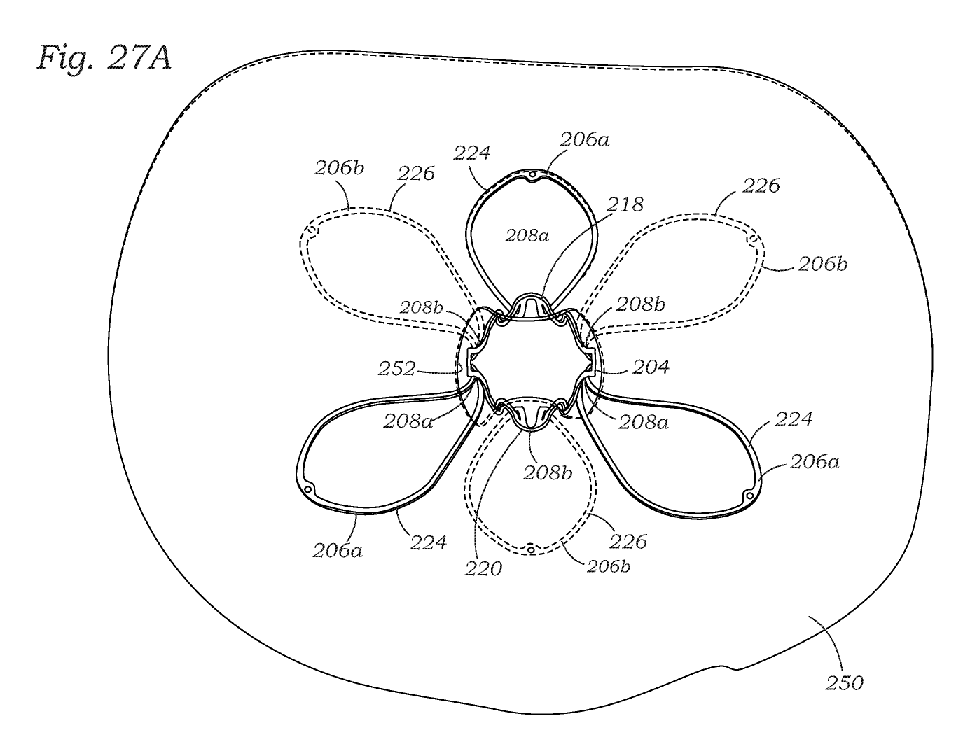

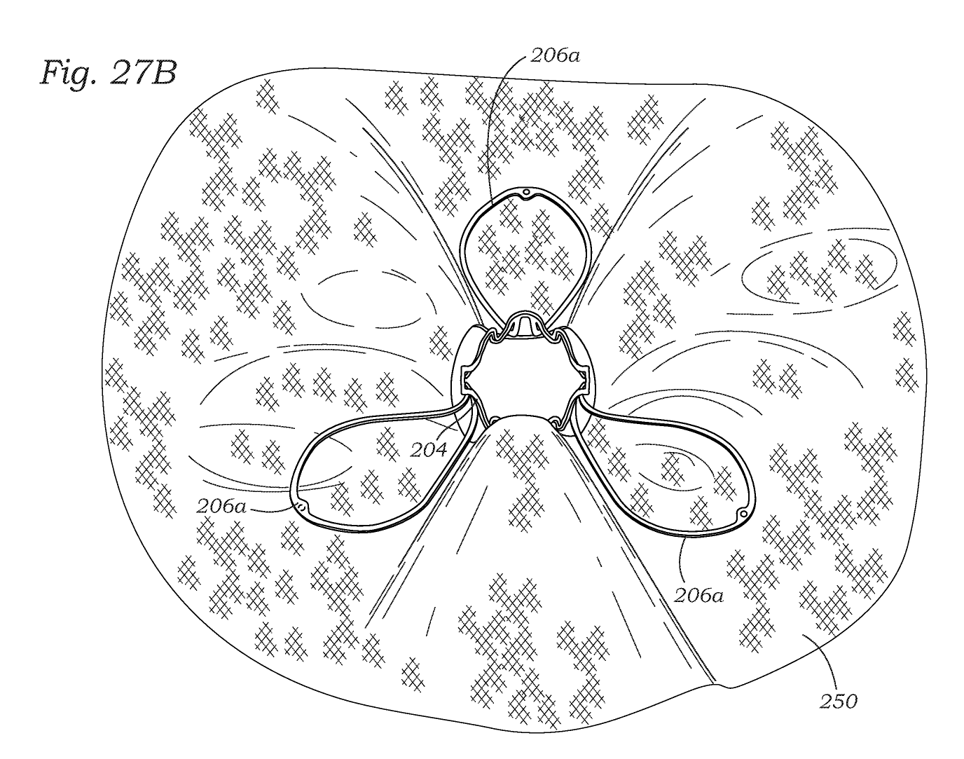

FIGS. 27A and 27B show the closure device 200 implanted within a septum 250 having an orifice 252. An occluding member 114 is not shown for purposes of illustration. The septum 250 can be, for example, the atrial septum of the heart. In the implanted position, the central portion 204 is positioned within the orifice 252. Each anchoring arm 206a is bent back from the second side 220 of the central portion toward its respective apex 208a on the first side 218 of the central portion such that the tissue engaging surface 222 presses against the adjacent surface of the septum 250. Likewise, each anchoring arm 206b is bent back from the first side 218 of the central portion toward its respective apex 208b on the second side 220 of the central portion such that the tissue engaging surface 226 presses against the adjacent surface of the septum 250 opposite the tissue engaging surfaces 222. As such, when closure device 200 is implanted in a patient, the anchoring arms will bear down on opposite sides of the patient's septal wall, thereby holding the closure device 200 securely in place with the central portion 204 in the orifice 252 in the septum 250.

The tissue secured between the anchoring arms holds the anchoring arms in their deflected state in which the anchoring arms 206a are held closer to the anchoring arms 206b compared to when the anchoring arms are in their expanded, non-deflected state (the gap G is reduced). If the thickness of the septum is approximately the size of the gap G in the non-deflected state, the anchoring arms 206a can be substantially co-planar with the anchoring arms 206b when the anchoring arms are in the deflected state. If the thickness of the septum is greater than the gap G in the non-deflected state, the anchoring arms are held in a position where the tissue engaging surfaces 222 face the tissue engaging surfaces 226.

One specific method for implanting the closure device 200 (and the other closure devices disclosed herein) within an atrial septum is as follows. Prior to insertion into the patient's body, the closure device 200 can be radially compressed to a radially compressed, delivery configuration and loaded into the distal end portion of a sheath of a delivery apparatus. In the radially compressed configuration, the anchoring arms 206a, 206b can be folded against the central portion 204 such that the tips of the anchoring arms 206a reside on the first side 218 of the central portion 204 and the tips of the anchoring arms 206b reside on the second side 220 of the central portion 204. Sutures can be threaded through eyelets 230 in one or more of the anchoring arms 206a, 206b and tensioned to fold the arms against the central portion 204. The sutures can be used later to control positioning and expansion of the anchoring arms at the implantation site. The sheath can be advanced distally over the closure device and/or an inner shaft of the delivery apparatus can be retracted proximally to draw the closure device 200 into the sheath.

Once loaded in the sheath, the delivery apparatus can be advanced percutaneously through the patient's vasculature to the right atrium of the heart in a trans-septal, antegrade approach for implanting the closure device 200 in the septum 250. In one approach, the delivery apparatus can be advanced through a femoral vein, the inferior vena cava, and into the right atrium. In another approach, the delivery apparatus can be advanced through a vein of the upper torso (e.g., a jugular vein), the superior vena cava, and into the right atrium.

Once in the right atrium, the delivery apparatus can be advanced through the septum 250 to position a distal end portion of the sheath in the left atrium. The sheath can then be retracted proximally to deploy the first set of arms 206a of the closure device 200, allowing the first set of arms 206a to radially expand within the left atrium. If sutures are used to retain the first arms 206a folded against the central portion, the user can gradually release tension on the sutures to allow the first arms 206a to expand in a controlled manner.

The entire delivery apparatus can then be retracted and/or otherwise positioned to bring the expanded first arms 206a against the septum 250 within the left atrium and to position the central portion 204 with in the orifice 252. The second arms 206b, still retained within the sheath, are located within the right atrium. The sheath can then be further retracted to expose the second arms 206b. If sutures are not used to retain the second arms in the compressed state, retraction of the sheath will allow the second arms 206b to radially expand within the right atrium and contact the septum 250 opposite the first arms 206a. If sutures are used, the user can gradually release tension on the sutures to allow the second arms 206b to expand in a controlled manner until they make contact with the septum 250. Expansion of the second arms 206b also allows the central portion 204 to expand radially within the orifice 252. Further details of a delivery apparatus and method for delivering a closure device are disclosed in co-pending U.S. Publication No. 2017/0224323 and co-pending U.S. Publication No. 2018/0333150, which are incorporated herein by reference.

Following implantation, the occluding member 114 (not shown in FIGS. 27A-27B) covers the orifice 252 and blocks the flow of blood. Overtime, atrial tissue can grow over the occluding member 114. If made from a bio-resorbable material, the occluding member 114 can degrade within the body and the re-grown atrial tissue can close the orifice 252. At any time following implantation of the closure device, the septal defect can be accessed for reentry through the defect by any means known to those skilled in the art with the benefit of the present disclosure.

In some embodiments, the occluding member 114 and/or regrown tissue can be punctured with a medical instrument (e.g., a catheter) if access through the septum 250 is needed in a subsequent procedure. If the medical instrument has a relatively small diameter, such as used for treating arrhythmias, the hole formed in the occluding member 114 and/or regrown tissue may be small enough to sufficiently inhibit blood flow between the left and right atriums without further intervention. If the medical instrument has a relatively large diameter, such as a delivery apparatus for implanting prosthetic valve, and leaves a relatively larger opening in the occluding member 114 and/or regrown tissue, another closure device can be implanted within the first device (e.g., device 200) to block blood flow between the right and left atriums.

Additionally, as noted above, the central portion 204 of the frame 202 can be expandable to accommodate entry of a medical instrument that has a larger diameter than the central portion 204 at rest (the “at rest” state being the shape-set configuration of the central portion). When the larger medical instrument is removed from the frame 202, the central portion 204 can revert back to its smaller, shape-set configuration under its own resiliency.

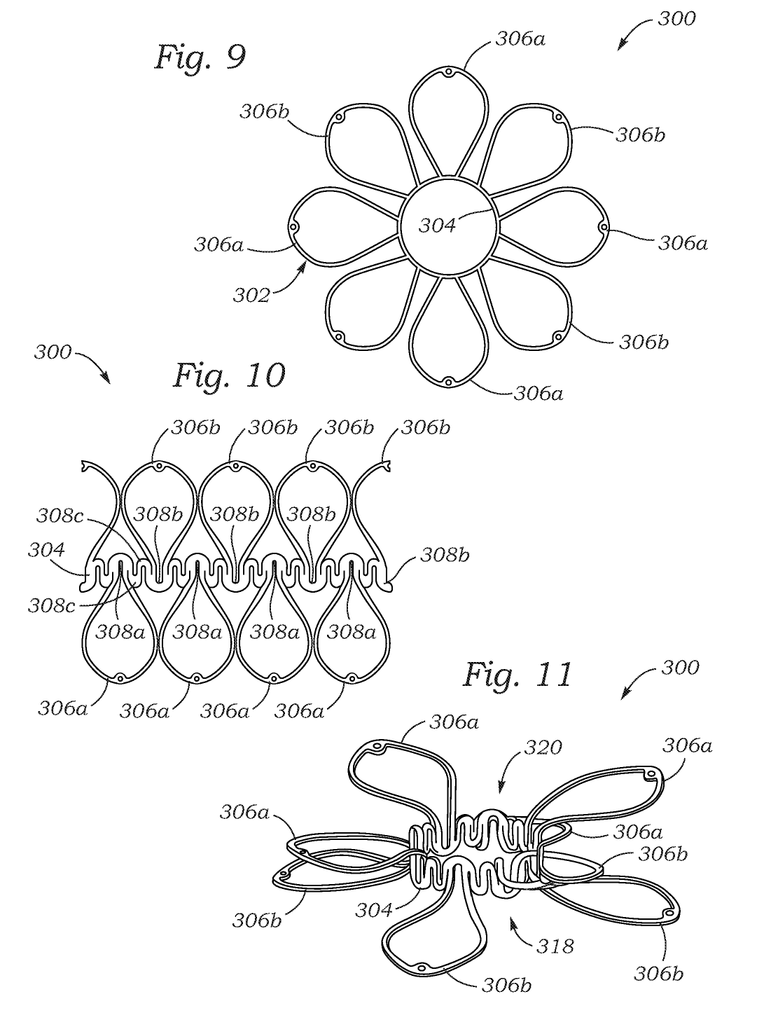

FIGS. 9-11 show an exemplary septal closure device 300, according to another embodiment. The septal closure device 300 comprises a frame 302 having a central portion 304, a first set of anchoring arms 306a, and a second set of anchoring arms 306b. Although not shown, an occluding member (e.g., occluding member 114) can be mounted on the frame 302 as described above in connection with closure device 100. The closure device 300 is constructed in a similar manner to the closure device 100 except that the closure device 300 has four anchoring arms in the first set of anchoring arms 306a and four anchoring arms in the second set of anchoring arms 306b. As can be seen in FIG. 10, the central portion 304 has a sinusoidal shape similar to the central portion 104. Each anchoring arm 306a, 306b extends from a respective apex 308a, 308b of the central portion 304. In the illustrated example, there are two apices 308c between each apex 308a and the closest apex 308b in the circumferential direction. In other examples, there can be any number of apices 308c between each apex 308a and the closest apex 308b.

As can best be seen in FIG. 11, the anchoring arms 306a extend from a first side 318 of the central portion 304 to a second side 320 of the central portion while the anchoring arms 306b extend from the second side 320 of the central portion to the first side 318 of the central portion. The closure device 300 can be implanted in the manner shown in FIGS. 27A and 27B such that the anchoring arms 306a bear against one side of the septum 250 and the anchoring arms 306b bear against the opposite side of the septum. As such, when closure device 300 is implanted in a patient, the anchoring arms will bear down on opposite sides of the patient's septal wall, thereby holding the closure device 300 securely in place with the central portion 304 in the orifice 252 in the atrial septum.

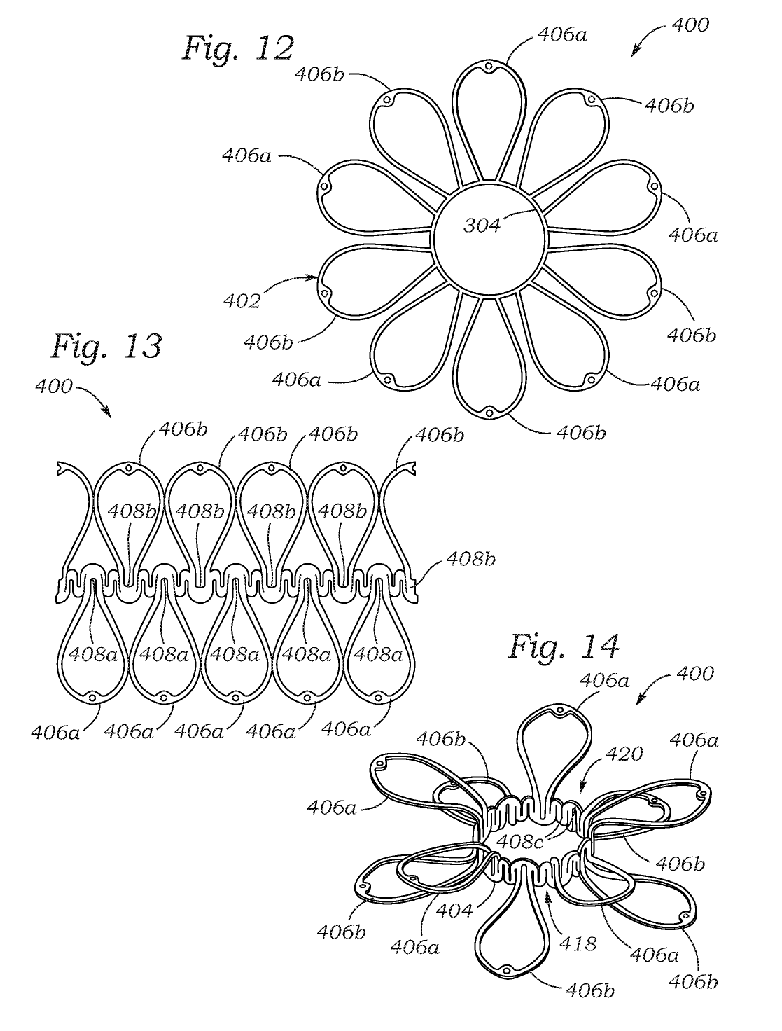

FIGS. 12-14 show an exemplary septal closure device 400, according to another embodiment. The septal closure device 400 comprises a frame 402 having a central portion 404, a first set of anchoring arms 406a, and a second set of anchoring arms 406b. Although not shown, an occluding member (e.g., occluding member 114) can be mounted on the frame 402 as described above in connection with closure device 100. The closure device 400 is constructed in a similar to the closure device 100 except that closure device 400 has five anchoring arms in the first set of anchoring arms 406a and five anchoring arms in the second set of anchoring arms 406b. As can be seen in FIG. 13, the central portion 404 has a sinusoidal shape similar to the central portion 104. Each anchoring arm 406a, 406b extends from a respective apex 408a, 408b of the central portion 404. In the illustrated example, there are two apices 408c between each apex 408a and the closest apex 408b in the circumferential direction. In other examples, there can be any number of apices 408c between each apex 408a and the closest apex 408b.

As can best be seen in FIG. 14, the anchoring arms 406a extend from a first side 418 of the central portion 404 to a second side 420 of the central portion while anchoring arms 406b extend from the second side 420 of the central portion to the first side 418 of the central portion. The closure device 400 can be implanted in the manner shown in FIGS. 27A and 27B such that the anchoring arms 406a bear against one side of the septum 250 and the anchoring arms 406b bear against the opposite side of the septum. As such, when closure device 400 is implanted in a patient, the anchoring arms will bear down on opposite sides of the patient's septal wall, thereby holding the closure device 400 securely in place with the central portion 404 in the orifice 252 in the atrial septum.

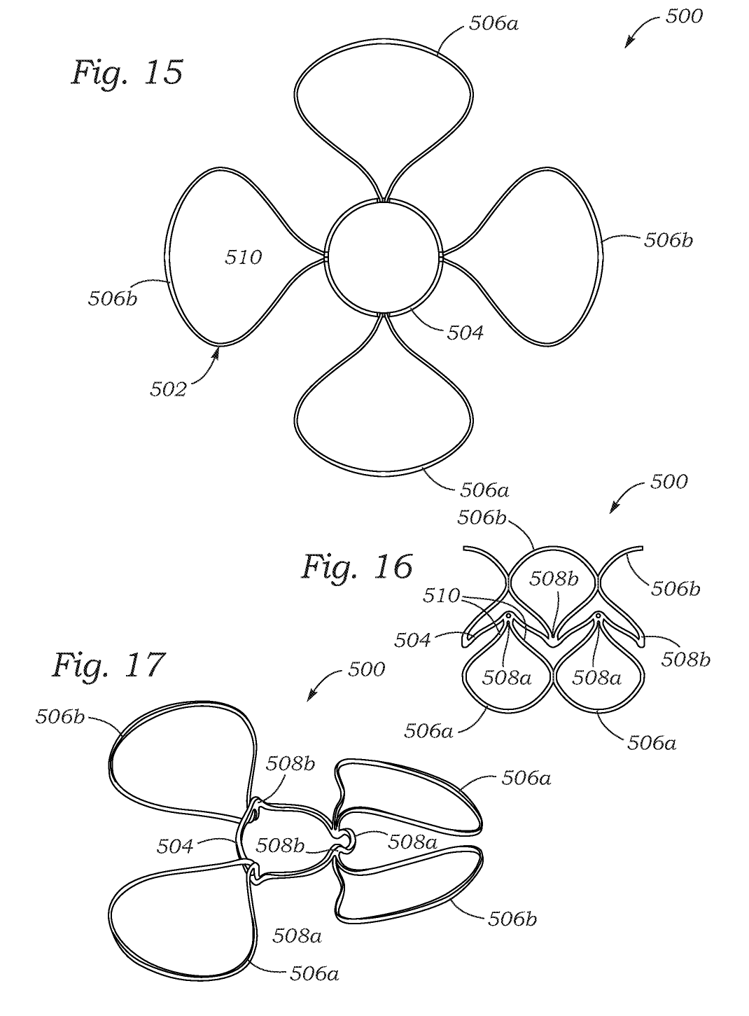

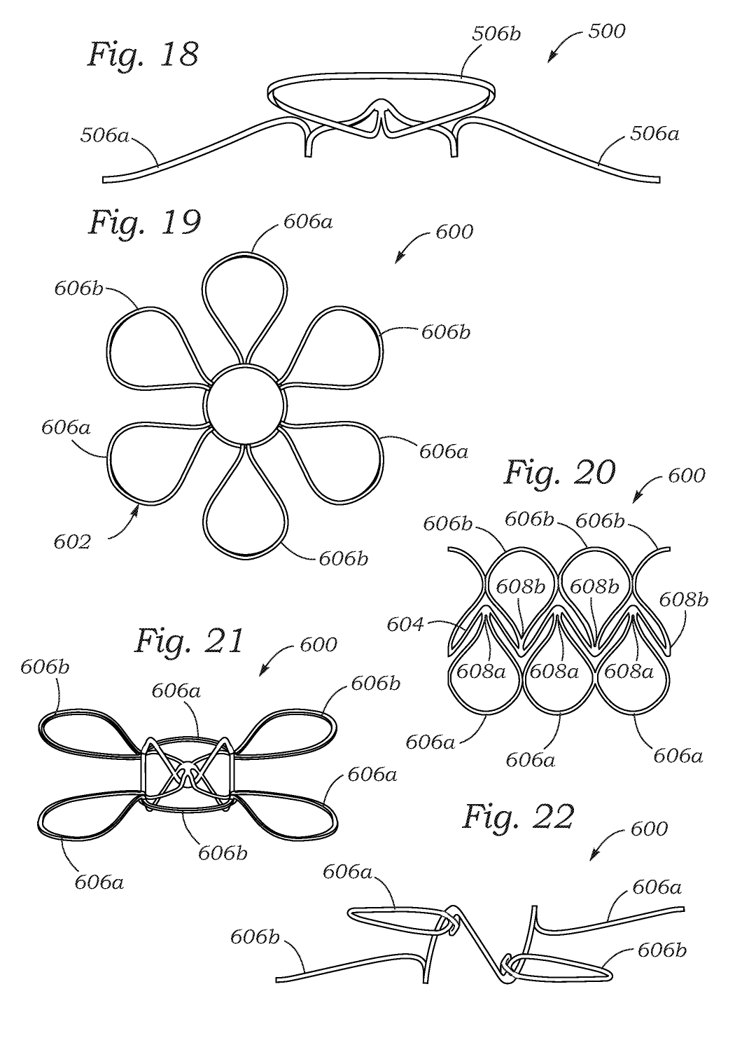

FIGS. 15-18 show an exemplary septal closure device 500, according to another embodiment. The septal closure device 500 comprises a frame 502 having a central portion 504, a first set of anchoring arms 506a, and a second set of anchoring arms 506b. Although not shown, an occluding member (e.g., occluding member 114) can be mounted on the frame 502 as described above in connection with closure device 100. The closure device 500 is constructed in a similar manner to the closure device 100 except that the anchoring arms 506a, 506b have a different shape than anchoring arms 106a, 106b and the central portion 504 has a different shape than the central portion 104.

In the example of FIGS. 15-18, the connection locations where the two legs 510 of each anchoring arm 506a, 506b are connected to the central portion 504 are not spaced apart, whereas in the example of FIGS. 1-4, the connection locations where the two legs 130 of each anchoring arms 106a, 106b are connected to the central portion 104 are spaced apart. Thus, the two legs 510 of each anchoring arm can converge and contact the central portion at the same location. Additionally, as can best be seen in FIGS. 16-18, the central portion 504 has a number of apices equal to the number of anchoring arms (four total anchoring arms and four total apices in the example closure device 500). This is in contrast to closure device 100, in which there are more apices than anchoring arms (apices 108c are not connected to any corresponding anchoring arms). As can be seen in FIG. 16, each anchoring arm 506a, 506b extends from a respective apex 508a, 508b of the central portion 304.

FIGS. 19-22 show an exemplary septal closure device 600, according to another embodiment. The septal closure device 600 comprises a frame 602 having a central portion 604, a first set of anchoring arms 606a, and a second set of anchoring arms 606b. Although not shown, an occluding member (e.g., occluding member 114) can be mounted on the frame 602 as described above in connection with closure device 100. The closure device 600 is constructed in a similar manner to the closure device 500 except that first set of anchoring arms 606a and the second set of anchoring arms 606b each comprise three anchoring arms. As can be seen in FIG. 20, the central portion 604 has a shape similar to the central portion 504. Each anchoring arm 606a, 606b extends from a respective apex 608a, 608b of the central portion 604.

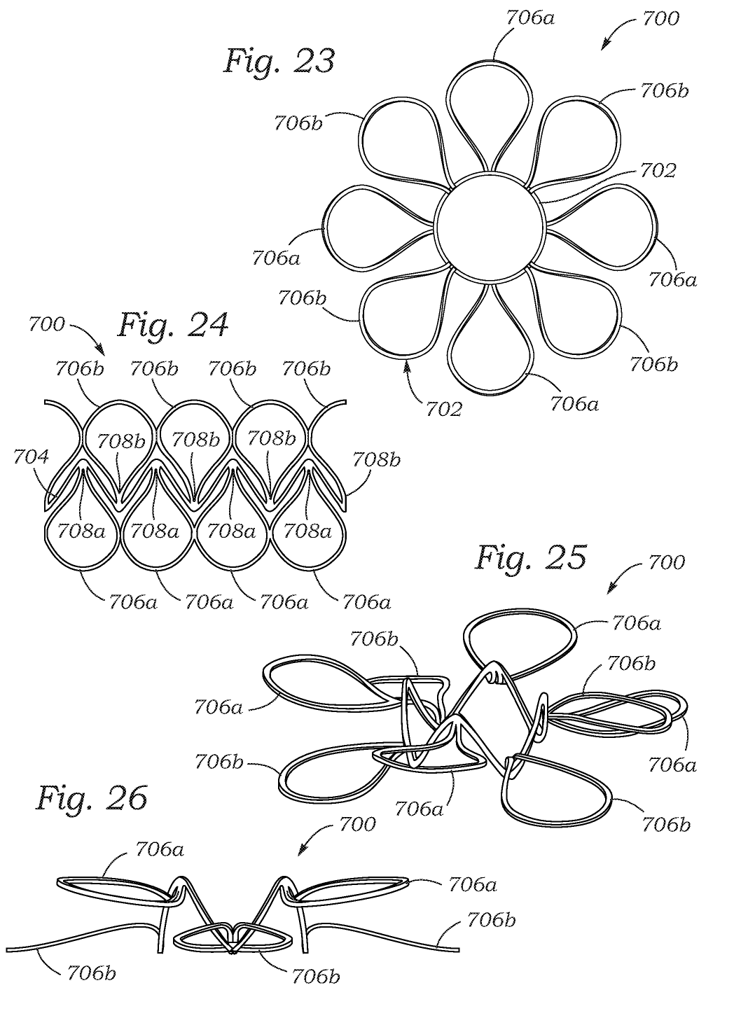

FIGS. 23-26 show an exemplary septal closure device 700, according to another embodiment. The septal closure device 700 comprises a frame 702 having a central portion 704, a first set of anchoring arms 706a, and a second set of anchoring arms 706b. Although not shown, an occluding member (e.g., occluding member 114) can be mounted on the frame 702 as described above in connection with closure device 100. The closure device 700 is constructed in a similar manner to the closure device 500 except that the first set of anchoring arms 706a and the second set of anchoring arms of 706b each comprise four anchoring arms. As can be seen in FIG. 24, the central portion 704 has a shape similar to the central portion 504. Each anchoring arm 706a, 706b extends from a respective apex 708a, 708b of the central portion 604.

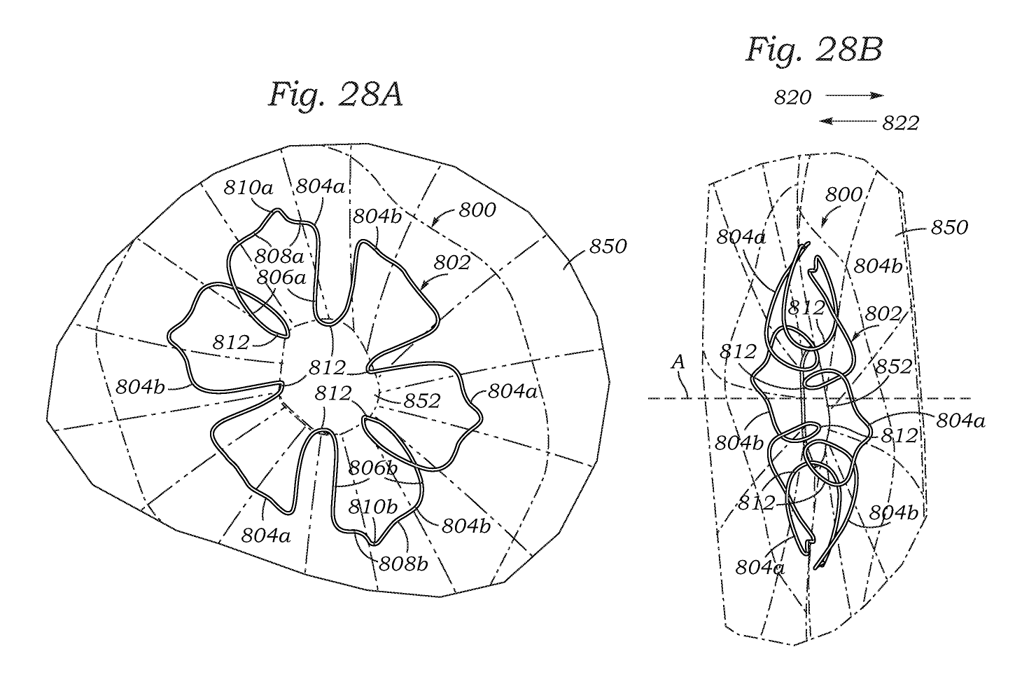

FIGS. 28A-28B show an exemplary septal closure device 800, according to another embodiment. The septal closure device 800 comprises a frame 802. In particular embodiments, the frame 802 can comprise a closed loop wire-form that can be formed from a single wire that is bent into the shape shown in FIGS. 28A-28B. In other embodiments, the frame 802 can be cut (e.g., laser cut) from a blank (e.g., a flat or tubular piece of material) and then shape set in the form shown. The frame 802 can be formed from any of the materials described above in connection with the frames of the previously described embodiments. The device 800 can include an occluding member as previously described.

The frame 802 can have a first set of anchoring arms 804a and a second set of anchoring arms 804b. Each of the arms 804a in the illustrated embodiment can have two side portions 806a, two upper portions 808a extending toward each other from respective radial outer ends of the side portions 806a, and a tip 810a formed between the upper portions 808a. In the illustrated example of FIG. 28A, the side portions 806a are bent slightly away from each other as they extend radially outwardly from their radial inner ends. In other examples, the side portions 806a can be substantially parallel or can be bent towards each other moving in a direction outwardly from their radial inner ends. The upper portions 808a can be substantially perpendicular to the side portions 806a. The tip 810a can be U-shaped, as shown in FIG. 28A. In some examples, the tip 810a is not present in the arms 804a and the upper portions 808a comprise a straight section extending between the outer ends of the side portions 806a. Each of the arms 804b can be constructed in a similar manner to the arms 804a and can comprise two side portions 806b, two upper portions 808b, and a tip 810b.

In the illustrated example, there are three anchoring arms 804a and three anchoring arms 804b. In other examples, there can be any number of anchoring arms 804a and 804b. Each anchoring arm 804a can be connected to two adjacent anchoring arms 804b by two U-shaped portions 812. Each U-shaped portion 812 extends from a side portion 806a of the arm 804a to an adjacent side portion 806b of an adjacent arm 804b. As best shown in FIG. 28B, each U-shaped portion 812 extends generally axially as it extends from a side portion 806a of an arm 804a to a side portion 806b of an arm 804b. Thus, the shape of the U-shaped portion 812 spaces apart the radial inner ends of the side portion 806a and the side portion 806b in the axial direction (a direction parallel to a central axis A extending through the center of the frame).

As can best be seen in FIG. 28B (which shows the device 800 implanted in a septum), the arms 804a are bent or curved in a first direction 820 toward the arms 804b and the arms 804b are bent or curved in a second direction 822 toward the arms 804a. Accordingly, when the closure device 800 is implanted in a septum 850 having an orifice 852, the first set of anchoring arms 804a press against a first side of the septum and the second set of anchoring arms 804b press against a second side of the septum to hold the closure device, as discussed in more detail below.

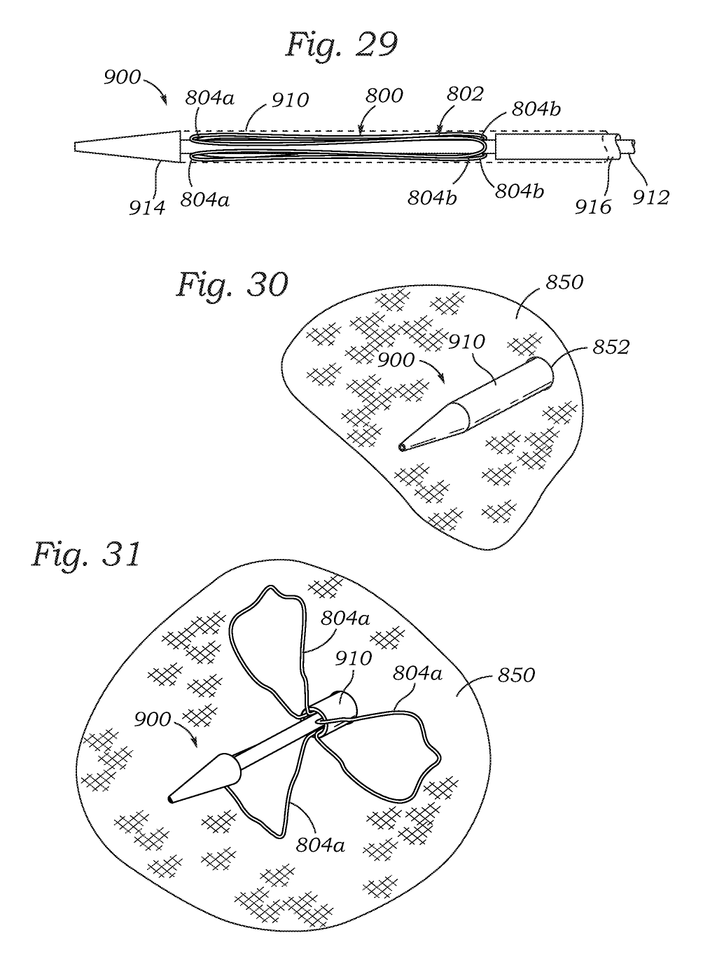

The frame 802 can be configured such that the anchoring arms 804a, 804b can move from a non-deflected state prior to implantation in a septum, as shown in FIG. 29, to a deflected state when implanted in the septum, as shown in FIGS. 28A, 28B. The frame 802 can be made from a shape-memory material such as Nitinol, and can be shape set in the shape shown in FIGS. 28A, 28B. As such, during an implantation procedure, discussed below, the frame 802 naturally assumes the shape of FIGS. 28A, 28B. Similar to the previously described embodiments, the central portion of the frame defined by the U-shaped portions 812 can expand radially to accommodate a larger medical device that is inserted through the closure device 800 in a subsequent procedure.

One specific method for implanting the closure device 800 is as follows. Prior to insertion into a patient's body, the closure device 800 can be placed within a sheath 910 of a delivery apparatus 900 in a radially compressed state, as shown in FIG. 29. The delivery device 900 can include an inner shaft 912 that extends coaxially through the sheath 910 and a nose cone 914 mounted on the distal end of the inner shaft 912. The delivery apparatus 900 can further include a shaft 916 that extends coaxially between the inner shaft 912 and the sheath. The shaft 916 can serve as a pushing member and/or a retaining member for releasably retaining the closure device 800 during deployment. The delivery apparatus 900 can be advanced through a patient's vasculature and through the orifice 852 in the septum 850, as shown in FIG. 29. The sheath 910 can then be retracted to expose the anchoring arms 804a, causing them to radially expand as shown in FIG. 31.

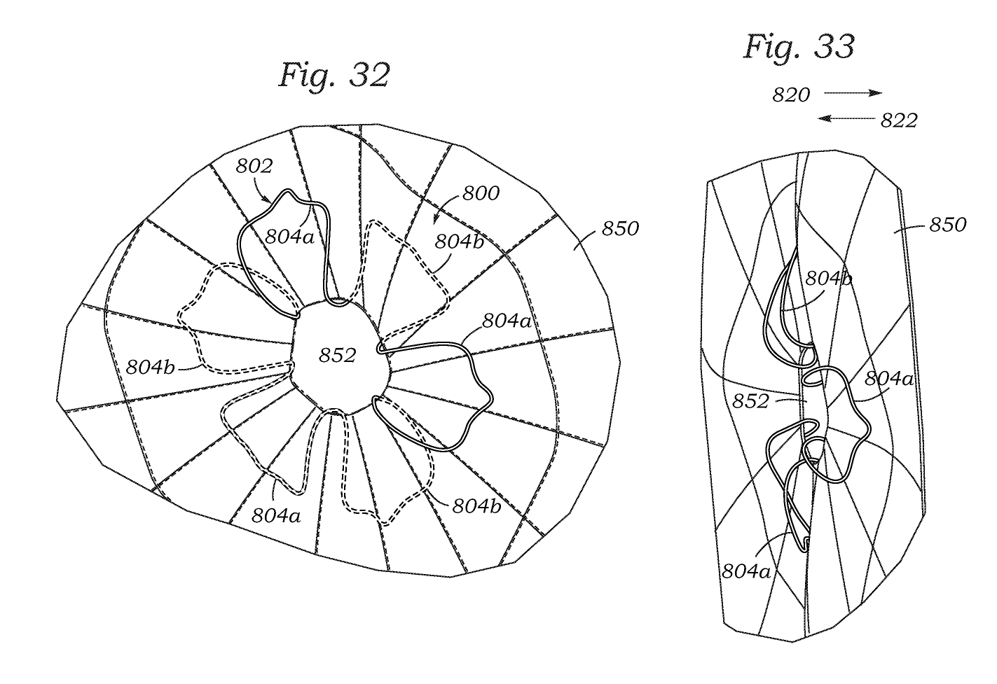

After deployment of the arms 804a, the entire delivery apparatus 900 can be retracted to bring the arms 804a against the adjacent surface of the septum. The sheath 910 can then be further retracted to expose the anchoring arms 804b, causing them to radially expand and contact the opposite side of the septum, as shown in FIG. 32. In this radially expanded state, the arms 804a press against a first side of the septum 850 and the arms 804b press against a second side of the septum, thereby holding the frame 802 securely in place.

In alternative embodiments, any of the frames described above in connection with FIGS. 1-33 (e.g., any of frames 102, 202, 302, 402, 502, 602, 702, 802), when provided without a blood-occluding member, can be used to maintain the patency of an orifice in tissue and therefore can be used as a shunt to permit fluid (e.g., blood) to flow through the orifice. For example, a frame (e.g., any of frames 102, 202, 302, 402, 502 602, 702, 802) can be implanted in an orifice in the atrial septum to allow blood to flow from the left atrium into the right atrium to reduce pressure in the left atrium, which can help treat pulmonary hypertension. Eliminating the blood-occluding member inhibits healing of the orifice and instead the frame functions to maintain an opening between the atria sufficient to reduce blood pressure in the left atrium.

In particular embodiments, a method of treating pulmonary hypertension comprises forming an orifice in the atrial septum (e.g., a 7-9 mm orifice) using a needle inserted through the vasculature of a patient (e.g., through the inferior or superior vena cava) and into the right atrium of the heart. The end of the needle is used to puncture the atrial septum and form the orifice. Thereafter, a shunt comprising any of frames 102, 202, 302, 402, 502, 602, 702, 802 (or any of the modifications of these frames described above) can be implanted in the orifice, such as using the delivery apparatus and the method described above corresponding to the delivery apparatus.

### GENERAL CONSIDERATIONS

For purposes of this description, certain aspects, advantages, and novel features of the embodiments of this disclosure are described herein. Features, integers, characteristics, compounds, chemical moieties or groups described in conjunction with a particular aspect, embodiment or example of the disclosure are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods.

As used herein, the terms “a”, “an”, and “at least one” encompass one or more of the specified element. That is, if two of a particular element are present, one of these elements is also present and thus “an” element is present. The terms “a plurality of” and “plural” mean two or more of the specified element.

As used herein, the term “and/or” used between the last two of a list of elements means any one or more of the listed elements. For example, the phrase “A, B, and/or C” means “A”, “B,”, “C”, “A and B”, “A and C”, “B and C”, or “A, B, and C.”

As used herein, the term “coupled” generally means physically coupled or linked and does not exclude the presence of intermediate elements between the coupled items absent specific contrary language.

In view of the many possible embodiments to which the principles of the disclosed technology may be applied, it should be recognized that the illustrated embodiments are only preferred examples of the disclosure and should not be taken as limiting the scope of the disclosure. Rather, the scope of the disclosure is defined by the following claims. I therefore claim as my disclosure all that comes within the scope and spirit of these claims.

We claim:

1. An implantable closure device comprising:
a self-expanding metal frame comprising a central portion defining a lumen and a plurality of first anchoring arms and a plurality of second anchoring arms angularly spaced around the central portion;
wherein the central portion comprises a sinusoidal-shaped ring defining a plurality of apices;
wherein the first anchoring arms are connected to locations inside of the apices on a first side of the central portion and the second anchoring arms are connected to locations inside of the apices on a second side of the central portion;
wherein the first anchoring arms are configured to bear against a first surface of a septal wall; and
wherein the second anchoring arms are configured to bear against a second surface of the septal wall.

2. The closure device of claim 1, having at least two first anchoring arms and at least two second anchoring arms.

3. The closure device of claim 1, wherein the sinusoidal-shaped ring comprises a plurality of angled struts interconnected by the plurality of apices.

4. The closure device of claim 1, wherein the sinusoidal-shaped ring comprises a first set of apices connected to the first anchoring arms, a second set of apices connected to the second anchoring arms, and a third set of apices that are not connected to either the first anchoring arms or the second anchoring arms.

5. The closure device of claim 4, wherein there is at least one apex of the third set of apices positioned circumferentially between each first anchoring arm and a second anchoring arm.

6. The closure device of claim 1, wherein the sinusoidal-shaped ring comprises a number of apices equal to the total number of first anchoring arms and second anchoring arms.

7. The closure device of claim 1, further comprising an occluding member mounted on the frame and at least partially covering the lumen of the central portion.

8. The closure device of claim 7, wherein the occluding member is configured to be pierced by a medical instrument.

9. The closure device of claim 7, wherein the occluding member comprises a fabric.

10. An implantable closure device comprising:
a self-expanding metal frame comprising a central portion defining a lumen and a plurality of first anchoring arms and a plurality of second anchoring arms angularly spaced around the central portion;
wherein the first anchoring arms and the second anchoring arms each have tissue engaging surfaces;
wherein the first anchoring arms and the second anchoring arms are configured to move from a non-deflected state prior to implantation in a septum to a deflected state when implanted in the septum, wherein when the anchoring arms are in the non-deflected state, the tissue engaging surfaces of the first anchoring arms and the tissue engaging surfaces of the second anchoring arms face away from each other, and wherein when the anchoring arms are in the deflected state, the tissue engaging surfaces of the first anchoring arms and the tissue engaging surfaces of the second anchoring arms contact opposing sides of the septum.

11. The closure device of claim 10, having at least two first anchoring arms and at least two second anchoring arms.

12. The closure device of claim 10, wherein the central portion comprises a sinusoidal-shaped ring defining a plurality of apices and each anchoring arm extends from one of the apices.

13. The closure device of claim 10, wherein each anchoring arm has a first portion extending from the central portion generally parallel to a central axis of the closure device and a second portion extending radially outwardly from the first portion.

14. The closure device of claim 10, wherein the sinusoidal-shaped ring comprises a first set of apices connected to the first anchoring arms, a second set of apices connected to the second anchoring arms, and a third set of apices that are not connected to either the first anchoring arms or the second anchoring arms.

15. The closure device of claim 10, further comprising an occluding member mounted to the frame and at least partially covering the lumen of the central portion.

16. A method of implanting a closure device comprising:
coupling the closure device to a distal end portion of a delivery apparatus, wherein the closure device comprises a self-expanding metal frame comprising a central portion defining a lumen and a plurality of first anchoring arms and a plurality of second anchoring arms angularly spaced around the central portion, wherein the first anchoring arms and the second anchoring arms each have tissue engaging surfaces, wherein the frame has a non-deflected state in which the tissue engaging surfaces of the first anchoring arms and the tissue engaging surfaces of the second anchoring arms face away from each other;
inserting the distal end portion of the delivery apparatus and the closure device into a patient's body;
positioning the central portion of the closure device within a septal orifice of a septal wall; and
positioning the first anchoring arms and the second anchoring arms such that the tissue engaging surfaces of the first anchoring arms engage a first side of the septal wall and the tissue engaging surfaces of the second anchoring arms engage a second side of the septal wall such that the first and second anchoring arms are held in a deflected state.

17. The method of claim 16, wherein the closure device is held in a radially compressed state when inserted into the patient's body and is radially expanded prior to or during the acts of positioning.

18. The method of claim 16, wherein the central portion comprises a sinusoidal-shaped ring defining a plurality of apices and each anchoring arm extends from the inside of one of the apices.

19. The method of claim 16, wherein the first anchoring arms are spaced from the second anchoring arms by a gap when the anchoring arms are in the non-deflected state and the gap is reduced when the anchoring arms are in the deflected state.

20. The method of claim 16, wherein the first and second anchoring arms are substantially co-planar with each other in the deflected state.
